(19) United States
(12) Patent Application Publication
(10) Pub. No.: US 2020/0000750 A1
(43) Pub. Date: Jan. 2, 2020

(54) Composition for Treating Acute and Chronic Pain

(71) Applicants: William MIDIAN, Silverlake, OH (US); Garin C. HOOVER, Sarasota, FL (US); Barbara D. WAMIL, Sarasota, FL (US)

(72) Inventors: William MIDIAN, Silverlake, OH (US); Garin C. HOOVER, Sarasota, FL (US); Barbara D. WAMIL, Sarasota, FL (US)

(21) Appl. No.: 16/449,676

(22) Filed: Jun. 24, 2019

Related U.S. Application Data
(60) Provisional application No. 62/690,603, filed on Jun. 27, 2018.

(51) Int. Cl. A61K 31/167; A61K 31/165; A61K 9/00

(52) U.S. Cl. CPC A61K 31/167; A61K 9/0053; A61K 31/165

(57) ABSTRACT

This invention is directed to methods and pharmaceutical compositions to improve the therapeutic benefit of combining colchicine and acetaminophen in the treatment of acute and chronic pain. This invention comprises a composition that effectively inhibits pain. The pharmaceutical composition can be administered orally, as a suppository, transcutaneously, or intravenously.

### CROSS-REFERENCE TO RELATED APPLICATIONS

This application claims the benefit of U.S. Provisional Application No. 62/690,603, filed Jun. 27, 2018, the entire contents of which is hereby fully incorporated herein by reference.

### TECHNICAL FIELD

The subject matter disclosed herein is generally directed to a combination of an effective amount of two compounds, colchicine, or derivatives thereof, and acetaminophen or derivatives thereof. This invention is concerned generally with the alleviation of pain and is particularly directed to methods and compositions to improve the therapeutic benefit of combining colchicine and acetaminophen in the treatment of acute and chronic pain. This invention comprises a composition that effectively inhibits pain. More specifically, colchicine and acetaminophen have been shown to treat acute pain in various diseases. This composition can be administered orally, as a suppository, transcutaneous, or intravenously.

### BACKGROUND OF THE INVENTION

Pain is the most common symptom for which patients seek medical assistance and relief, and chronic pain is among the most vexing problems that physicians face. In general, pain has two aspects: the first is a non-emotional perception of a stimulus or event which is usually strong enough to produce tissue damage to the person; the second is the individual's personal response to the perception of that stimulus or event. Pain implies damage to the patient, whether physical or psychological; and chronic pain, if untreated, will itself cause damage to the body.

Pain is sensed through the afferent pain pathway (Argoff C. Mechanisms of pain transmission and pharmacologic management. Curr Med Res Opin 2011; 27:2019, Lewis K S, Whipple J K, Michael K A, Quebbeman E J. Effect of analgesic treatment on the physiological consequences of acute pain. Am J Hosp Pharm 1994; 51:1539). Multiple cortical and subcortical structures are involved in the experience of pain. Recent tissue damage due to illness, injury, or surgery initiates the release of local inflammatory mediators (e.g., bradykinin, substance P, prostaglandins, potassium, histamine, and serotonin). These mediators may cause primary hyperalgesia (augmented sensitivity to painful stimuli) or allodynia (misperception of pain with stimuli that are not noxious). Increased excitability of neurons in the central nervous system due to glutamate activation of the spinal N-methyl-D-aspartate receptors may exacerbate pain perception (secondary hyperalgesia). Patients with a preexisting chronic pain syndrome, neuropathy, or myopathy may develop exacerbation of baseline pain due to hyperalgesia or allodynia.

Analgesic agents and techniques are used to reduce pain by:
Altering perception of pain in the central nervous system (e.g., opioid analgesics, acetaminophen);
Inhibiting local production of pain mediators (e.g., blockage of prostaglandin synthesis by nonsteroidal antiinflammatory drugs [NSAIDs]);
Interrupting neural impulses in the spinal cord (e.g., local anesthetic agents used for a neuraxial block).

Pain is also clinically identified as being either acute or chronic. A common view holds that the difference between acute and chronic pain can be described by the duration of the pain. Pain lasting over six months in duration is typically considered chronic; and any shorter time period of pain is usually considered acute. Several other clinical features are also traditionally used to differentiate acute pain from chronic pain. Patients suffering from severe acute pain often give a clear description of its location, character, and timing. Also, acute pain usually responds well to analgesic agents; and the psychological makeup of the patient often plays only a minor role in the pathogenesis. In contrast, patients suffering from chronic pain typically are unable to describe precisely the location, character, and timing of the pain. Furthermore, chronic pain often is less responsive to analgesic agents; and the individual's psychologic state has a larger role. The clinician's dilemma is increased, since there are no reliable, objective tests by which to assess chronic pain. For these reasons, the physician normally accepts his patient's report, taking into consideration his age, cultural background, environment, and other psychologic background factors known to alter a person's subjective reaction to pain.

Physicians also conventionally divide chronic pain into three somewhat overlapping categories in decreasing order of frequency. These are: psychophysiological disorders; chronic pain associated with structural disease; and somatic delusions.

Chronic pain associated with structural disease may be characterized by prolonged episodes of pain such as occurs with rheumatoid arthritis, metastatic cancer, or sickle cell anemia. The patient may have prolonged episodes of pain alternating with pain-free intervals; or display unremitting pain, which varies in severity. Psychological factors may play an important role in increasing or relieving pain, but the treatment of the chronic pain by analgesics or correcting the underlying disease is typically more helpful.

The symptomatic management of pain is dependent upon its severity and cause. The relief of chronic pain is often perplexing and difficult because useful measures in the treatment of acute pain are often ineffective for chronic pain. It is often necessary to resort to a combination of indirect and multidisciplinary therapeutic methods in order to provide any sort of relief whatsoever.

For all these reasons, it will be recognized that the combination of colchicine and acetaminophen to provide effective relief to a patient afflicted with acute or chronic pain is seen as an advance in therapeutic methods.

Colchicine is recognized as an anti-inflammatory agent, which is pharmacologically effective against gouty arthritis. It provides dramatic relief of acute attacks of gout and is an effective prophylactic agent against such attacks. It is not recognized as an analgesic and does not provide relief of other types of pain. Colchicine is also employed as a research tool via its capability as an antimitotic agent. The mechanism of action may interfere with intracellular assembly of inflammasome complex present in neutrophils and monocytes, which mediates activation of interleukin-1β. It is widely employed as an experimental tool in the study of normal and abnormal cell division and cell function.

Aside from its use for the relief of acute attacks of gout, colchicine is employed because it can arrest plant and animal cell division both in-vitro and in-vivo. Mitosis is arrested in the metaphase, due to failure of spindle formation. Bizarre and often abnormal nuclear configurations ensue and the cells then often die.

Other published effects of colchicine include: the ability to inhibit the relief of histamine-containing granules from mast cells under varying conditions; the ability to lower body temperature; the ability to increase the sensitivity of the body to central depressants; and the ability to depress the respiratory center.

### Colchicine and its Analogs

### The Naturally Occurring Compounds

Although the exact formula of colchicine, one of the purified ingredients of colchicum, is now well established, its actual synthesis in the laboratory has not been accomplished. We still must obtain our materials from the natural source. There are 64 varieties of the plant colchicum (B. Stefanoff, 1928), all members of the lily family. At least 30 contain colchicum. In the western world, the source is mainly the corm and seed of Colchicum autumnale, a plant found growing wild in the Mediterranean basin. Many other varieties are found further west, including the Himalayan Mountains. Briefly, colchicine is extracted from the corm and seed with alcohol and water, and then from this solution with chloroform. The latter is evaporated off, yielding amorphous colchicine, which can be crystallized from solution in ethyl acetate. Chromatographic purification is then accomplished by the method of Ashley and Harris (1944). This yields practically pure colchicine. Santavy et al. have extracted at least nine other analogs naturally present in the plant itself (Santavy et al. 1951). From these basic natural compounds, a host of synthetically derived analogs have been developed, mainly for experimental use in cancer. Some of these have been applied to the treatment of gout and will be referred to below.

### The Structural Formula: Tropolone

The formula proposed for colchicine by Dewar in 1945 is now generally accepted. It has been shown to be a three-ring structure: ring A containing six carbons with three methoxy groups, ring B is seven membered, containing an acetylamide group, and ring C also is seven membered, containing a meth-oxy and ketone group. The basic structure, therefore, of rings B and C is troponoloid. The chemistry of tropolone and its derivatives has been elucidated by Dewar (1945), Cook and Loudon (1951) and others. To date, tropolone has been found in cypress, the members of the lily family, and in Penicillium puberulum. It is not a spindle poison but rather favors the formation of the spindle. It is a potent antagonist of colchicine, along with mesoinosital, a well known insecticide (Benitez et al. 1954). Perhaps the presence of tropolone in the plant explains the lack of toxic effect of colchicine in the living plant, similar to the neutralization of venom in a living snake. Phlorizine, on the other hand, is a synergist of colchicine, increasing its antimitotic effect eight times (H. R. Littre, et al. 1951).

### Effect of Colchicine Analogs in Gout

In considering the more recent research on the newer analogs that might be effective in acute gout, an important factor is that toxicity, antimitotic activity, the inhibition of tumor growth, and the anti-gout effects, are definitely not parallel. Therefore, in seeking a better anti-gout drug, it is obvious that each new analog may have to be separately studied. Boiling colchicine with dilute hydrochloric acid yields colchiceine, which is both a very weak antimitotic and diarrheal, and has no anti-gout effects in doses given (5 to 8 mg. orally). Further hydrolysis yields trimethylcolchicinic acid, first produced by Zeisel as long ago as 1887, and now particularly prominent as a possible improvement over colchicine in the treatment of gout. Colchiceine is an iso-colchicine, in that the positions of the ketone and methoxy groups in ring C are reversed and the methoxy replaced by a hydroxy group. Iso-colchicine and all other iso forms of the compounds have been found to be relatively ineffective as mitotic poisons, have little or no anti-gout effects and are relatively nontoxic. Contrariwise, various substitutions on ring C may markedly augment the antimitotic effects, and because of these facts it appears that an important factor in the antimitotic effect is the constitution of ring C. Also at least one methoxy group appears indispensable in ring A. Of the many naturally occurring colchicine analogs so far extracted and identified, and of the very many artificially derived analogs, most have been experimentally applied to the inhibition of tumor growth in animals and in tissue cultures, but very few have been used in the treatment of acute attacks of gout. The brief summary of the chemical constitution and present knowledge of those analogs tried so far in the management of acute attacks of gout has been covered in the paper by S. L. Wallace in October 1959.

Desacetylmethylcolchicine (DMC), also called colcimid and Demecolcin (Ciba), is the Compound F of Santavy, and is found naturally in the plant. It has had the most extensive trials in gout as well as in cancer and blood dyscrasias. It has been shown, in both animals and humans, to have a very depressing effect on granulocytopoiesis, much greater than colchicine. For this reason, it has been recommended and used fairly extensively in myelogenous leukemia. Because of these properties, it has dangerous potentialities as an anti-gout remedy. It also has produced temporary total alopecia in humans with the doses mentioned and, in birds, loss of feathers. Impairment of spermatogenesis has also been demonstrated. For all these reasons, it is not considered a satisfactory drug in the treatment of acute gouty attacks.

The desacetylthiocolchicine (DTC), in which SCH3, a thiomethyl group, replaces the methoxy group in ring 3, and an amino group the acetamide in ring 2, and colchicoside in which a glucoside replaces the methyl group in the first ring, have been shown to have equivocal results in acute gout.

The colchiceinamide (J. Leiter et al., 1952) substitutions in ring C produce the greatest antimitotic effect and have been extensively studied in cancer. They are more antimitotic than colchicine and in general less toxic. The hydroxy group of ring C of colchiceine, through the action of ammonia, results in its substitution by NH2 (Zeisel, 1888). The H1 or H2 of this radical can be variously substituted, yielding a large battery of compounds effective against experimental cancer. None of these have been used in gout as of yet.

Acetaminophen (USAN) or paracetamol (INN) (chemically known as N-(4-hydroxyphenyl)acetamide) is an antipyretic and analgesic commonly used to manage fever of any etiology, minor and severe pains (including post-operative pain) and a variety of aches. Acetaminophen acts on the hypothalamus to produce antipyresis. It may work peripherally to block pain impulse generation; and it may also inhibit prostaglandin synthesis in CNS. Acetaminophen is well tolerated and lacks many of the undesired effects of other analgesics, such as non-steroidal anti-inflammatory drugs (NSAIDs) or types of cyclooxygenase (COX) inhibitors (e.g., stomach lining irritation, adverse effects on platelets and renal function, fetal ductus aiterious closure complications and Reye's syndrome).

Acetaminophen is already approved to use in combination with one or more additional pharmaceutical agents. Those agents include opioids (natural, semi-synthetic, or synthetic), non-steroidal anti-inflammatory drugs (NSAIDs), benzodiazepines, barbiturates and other compounds, such as caffeine. Examples of compounds that were previously approved are: caffeine, morphine, hydrocodone, hydromorphone, levorphanol, aspirin, ketorolac, ibuprofen, naproxen, tramadol, dextropropoxyphene, methylhexital, diazepam, lorazepam, midazolam, propoxyphene, ketoprofen, flurbiprofen, etodolac, diclofenac, misoprostol, meloxicam, piroxicam, doxylamine, pamabrom, carisoprodol, and butalbital. One potential advantage of a combination formulation is that the formulation may induce analgesia beyond the ceiling effect of acetaminophen without approaching the toxic or nearly toxic dose levels of acetaminophen.

The new formulation of colchicine and acetaminophen will inhibit acute and chronic pain more potently than each drug alone.

### SUMMARY OF THE INVENTION

It is an object of the invention to effectively treat chronic and/or acute pain in mammals, and specifically in humans.

It is a further object of the invention to effectively treat chronic and/or acute pain in adult and pediatric patients with gout or rheumatoid arthritis.

These and other aspects, objects, features, and advantages of the example embodiments will become apparent to those having ordinary skill in the art upon consideration of the following detailed description of illustrated example embodiments.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains.

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview of Invention

The new formulation of the combination of colchicine and acetaminophen inhibits acute and chronic pain more potently than each drug alone.

Dose Modification—the preferred dose is between approximately 0.5 and 1.2 mg colchicine combined with between approximately 325 mg and 1 g acetaminophen, with a more preferred dose of between approximately 0.7 mg and 1 mg colchicine combined with between approximately 400 mg and 800 mg acetaminophen, with a more preferred dose of approximately 0.8 mg colchicine combined with approximately 600 mg of acetaminophen.

If original dose is 0.6 mg once or twice daily, it is possible to adjust the dose to 0.3 mg once or twice daily. The frequency and duration of administration of the colchicine-acetaminophen combination drug will depend on the condition being treated, the condition of the individual, and the like. The formulation may be administered to the individual one or more times, for example, 2, 3, 4, 5, 10, 15, 20, or more times. The formulation may be administered to the individual, for example, more than, equal to, or less than once a day, 2 times a day or 3 times a day. The formulation may also be administered to the individual, for example, less than once a day, for example, every other day, every third day, every week, or less frequently. The formulation may be administered over a period of days, weeks, or months.

Route of Administration—Oral, suppository, transcutaneous, or intravenous.

Schedule of Administration—Once a day (QD) or twice per day (BID). Maximum total dose of colchicine 1.2 mg per day and 1.3 g of acetaminophen.

Indications for Use—Pain, acute and chronic improvements for suboptimal chemotherapeutics including colchicine and acetaminophen thereof are made by the use of monitoring drug levels after dosing in an effort to maximize a patient's drug plasma level, to monitor the generation of toxic metabolites, or to monitor ancillary medicines that can be beneficial or harmful in terms of drug-drug interactions.

Colchicine Analogs—desacetylmethylcolchicine, colchiceinamide and colchiceinamide can be used in addition to colchicine in combination with acetaminophen.

Dosage Forms—Tablets, capsules, suppositories, powder or liquid. Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents, such as water. Such formulations may also comprise adjuvants, such as wetting agents, emulsifying and suspending agents, cyclodextrins, and sweetening, flavoring, and perfuming agents.

In some embodiments, the colchicine-acetaminophen combination can be administered parenterally, intravenously, or intramuscularly. Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. The sterile injectable preparation may also be a sterile powder to be reconstituted using acceptable vehicles prior to administration.

Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid may be used in the preparation of injectables.

The invention also includes formulations of the colchicine-acetaminophen combination administered in the form of suppositories for rectal administration. These can be prepared by mixing the agent with a suitable nonirritating excipient that is solid at room temperature but liquid at rectal temperature and, therefore, will melt in the rectum to release the drug. Such materials include cocoa butter, beeswax, and polyethylene glycols.

The colchicine-acetaminophen combination of the present invention may also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and/or metabolizable lipid capable of forming liposomes may be used. The present formulations in liposome form can contain, in addition to an acetaminophen prodrug, stabilizers, preservatives, excipients, and the like. In some embodiments, the lipids are phospholipids and/or phosphatidyl cholines (lecithins), natural and/or synthetic. Methods to form liposomes are known in the art (Prescott, Ed., Methods in Cell Biology, Volume XIV, Academic Press, New York, N.W., p. 33 1976).

The invention is further described in the following examples, which do not limit the scope of the invention described in the claims.

To demonstrate the efficacy and unexpected benefits of the present therapeutic methodology, individual case histories involving human patients are provided hereinafter. It will be expressly understood, however, that these individual case histories are merely representative of the unusual results and unique advantages provided by the present invention.

### Case Histories

### Case History 1

Patient 1 (“Patient 1”) was a 65 year old male with a fifteen year history of gout. The patient reported chronic pain at all times whether sitting, walking, or lying down. Patient 1 was administered colchicine (0.6 mg PO) and acetaminophen (650 mg) at first sign of flare. Patient 1 experienced an immediate reduction of pain and was markedly far more comfortable and relatively pain free one week later.

For 7-14 days duration, Patient 1 received 0.6 mg of colchicine and 650 mg of acetaminophen per day. Patient 1 experienced asymptomatic relief of pain; and remained pain-free after the passage of six weeks time.

### Case History 2

Patient 2 was a 63 year old female (“Patient 2”) with a 10 year history of chronic arthritic pain. Patient 2 reported chronic pain at all times. Patient 2 was administered colchicine (0.6 mg PO) and acetaminophen (325 mg) at first sign of flare. Patient 2 experienced an immediate reduction of pain and was markedly far more comfortable and relatively pain free one week later.

For 7-14 days duration, Patient 2 received 0.6 mg of colchicine and 325 mg of acetaminophen per day. Patient 2 experienced asymptomatic relief of pain; and remained pain-free after the passage of six weeks time.

Other medical conditions include, but are not limited to, chronic and acute pain that is associated with an inflammatory condition of a tissue or organ selected from skin, muscle, and joints, gout, pseudogout, neuropathic pain, ischemic injury (such as myocardial and/or cerebral), neuronal injury or a disease or condition that is responsive to the colchicine-acetaminophen combination.

To properly and completely understand the steps of the present therapeutic methodology, familiarity with current pharmacological therapies and medical modes of treatment for pain is needed. A summary review has been provided within the background subject matter previously disclosed herein. A more detailed review and minute description is provided by the following references:
Argoff C. Mechanisms of pain transmission and pharmacologic management. Curr Med Res Opin 2011; 27:2019
Lewis K S, Whipple J K, Michael K A, Quebbeman E J. Effect of analgesic treatment on the physiological consequences of acute pain. Am J Hosp Pharm, 51:1539, 1994
Joseph G. Chusid, Correlative Neuroanatomy & Functional Neurology, Lange Medical Publications, Los Altos, Calif., 1985
William F. Ganong, Review Of Medical Physiology 1985, Lange Medical Publications, Los Altos, Calif.
Crupp Chatton and Tierney, Current Medical Diagnosis & Treatment 1986, Lange Medical Publications, Los Altos, Calif.
Essentials Of Medicine, W. B. Saunders Company, 1986
P. Prithvi Raj, Practical Management Of Pain, 1986
Pain, Discomfort, and Humanitarian Care (Bonica, J. J., editor), Elsevier Publishing, New York, 1980
A Synopsis Of Anesthesia, 10th edition, 1987
Textbook Of Pain, (Wall and Melzack, editors), 2nd edition, 1989
The Management of Pain, (Bonica, J. J., editor), Lea and Febiger, 1953
Loeb J N. The influence of temperature on the solubility of monosodium urate. Arthritis Rheum. 15(2):189, 1972
Alvarellos A, Spilberg I. Colchicine prophylaxis in pseudogout. J Rheumatol. 13(4):804-805, 1986 [PubMed 3772928]
Becker M A. Treatment of calcium pyrophosphate crystal deposition (CPPD) disease. Post T W, ed. UpToDate. Waltham, Mass.: UpToDate Inc. http://www.uptodate.com. Accessed Mar. 15, 2018
Becker M A. Treatment of gout flares. Post T W, ed. UpToDate. Waltham, Mass.: UpToDate Inc. http://www.uptodate.com. Accessed May 10, 2018
Becker M A. Pharmacologic urate-lowering therapy and treatment of tophi in patients with gout. Post T W, ed. UpToDate. Waltham, Mass.: UpToDate Inc. http://www.uptodate.com. Accessed Mar. 14, 2018
Callen J P. Colchicine is effective in controlling chronic cutaneous leukocytoclastic vasculitis. J Am Acad Dermatol. 13(2, pt 1):193-200, 1985 [PubMed 4044947]
Callen J P, of Ekenstam E. Cutaneous leukocytoclastic vasculitis: clinical experience in 44 patients. South Med J. 80(7):848-851, 1987 [PubMed 3603105]
Colcrys (colchicine) [prescribing information]. Deerfield, Ill.: Takeda; December 2015.
Dalvi S R, Yildirim R, Yazici Y. Behcet's syndrome. Drugs. 2012; 72(17):2223-2241. doi: 10.2165/11641370-000000000-00000. [PubMed 23153327]
Das S K, Mishra K, Ramakrishnan S, et al. A randomized controlled trial to evaluate the slow-acting symptom modifying effects of a regimen containing colchicine in a subset of patients with osteoarthritis of the knee. Osteoarthritis Cartilage. 2002; 10(4):247-252. [PubMed 11950246]
Acetaminophen, 2017, https://en.wikipedia.org/wiki/Paracetamol
Acetaminophen--antiinflammatory, 2017, https://www.drugs.com/answers/tylenol-anti-inflammatory-drug-3002130.html
Dewar, M. J. S. Structure of colchicine. Nature 155141, 1945
Ashley, J. N. and Harris, J. O., Purification of colchicine by chromatography. J. Chem. Soc. 1944, p. 077.
Santavy F, Cenrnoch M, Malinsky J, Lang B, Zajickova A. Biol Listy. January 2; 31 (Suppl:75-84), 1951
The alkaloids: Chemistry and Pharmacology Volume 23, Pages 1-70, 1984
Wallace, S. L., Colchicine. Analogs in the treatment of acute gout. Arthr. & Rheumat. 5:389; 1959
Cook, J. W. and Loudon, J. D., The tropolones. Quart. Rev. 599, 1951
Doering, W. von E. and Knox, L. H., Tropolone. Jr. Am. Chem. Soc. 72:205, 1950; 73:428, 1951
Lettrk, H., Zur Chemie and Biologie der Mitosegiften, Angew. Chemie. 63:421, 1951
Uber Syngergisten von Mitosegiften V. Mitt. Versuche Zur Aufhebung der syngergistischen Wirkung durch Phosphagen. Natur-wiss. 38:13, 1951
Uber Mitosegifte. Ergebn. Physiol. 46-379, 1950

Various modifications and variations of the described methods, pharmaceutical compositions, and use of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of treating acute and chronic pain comprising administering to a patient in need of such treatment an effective amount of colchicine in combination with an effective amount of acetaminophen.

2. The method of claim 1, wherein the amount of colchicine administered is between approximately 0.6 mg and 1.2 mg once or twice a day, and the amount of acetaminophen is between approximately 325 mg and 1 g once or twice a day.

3. The method of claim 1 wherein the chronic and acute pain are associated with indications selected from gout, pseudogout, neuropathic pain, general ischemic injury, myocardial ischemic injury, cerebral and neuronal ischemic injury, rheumatoid arthritis, metastatic cancer, and sickle cell anemia.

4. The method of claim 2, wherein the amount of colchicine administered is between approximately 0.6 mg and 1.2 mg and the amount of acetaminophen is between approximately 325 mg and 1 g.

5. The method of claim 2, wherein the amount of colchicine administered is between approximately 0.7 mg and 1 mg and the amount of acetaminophen administered is between approximately 400 mg and 800 mg.

6. The method of claim 2, wherein the amount of colchicine administered is approximately 0.8 mg and the amount of acetaminophen administered is approximately 600 mg.

7. A pharmaceutical composition comprising colchicine and acetaminophen, or pharmaceutically acceptable salts thereof, in combination with a pharmaceutically acceptable carrier, wherein the colchicine and acetaminophen are each present in effective analgesic amounts.

8. The pharmaceutical composition of claim 7, wherein the composition is in a solid dosage form.

9. The pharmaceutical composition of claim 8, wherein the solid dosage form comprises an immediate release formulation.

10. The pharmaceutical composition of claim 8, wherein the solid dosage form comprises a controlled release formulation.

11. The pharmaceutical composition of claim 8, wherein the solid dosage form is a tablet, capsule or suppository.

12. The pharmaceutical composition of claim 7, wherein the composition is in a liquid oral dosage form.

13. The pharmaceutical composition of claim 7, wherein the composition is in a liquid intravenous or subcutaneous dosage form.
